(19) United States
(12) Patent Application Publication
(10) Pub. No.: US 2019/0216382 A1
(43) Pub. Date: Jul. 18, 2019

(54) PERSON MONITORING SYSTEM AND METHOD OF USE

(71) Applicant: Dan Baldwin, Henderson, CO (US)

(72) Inventor: Dan Baldwin, Henderson, CO (US)

(21) Appl. No.: 16/246,486

(22) Filed: Jan. 12, 2019

Related U.S. Application Data
(60) Provisional application No. 62/616,590, filed on Jan. 12, 2018.

(51) Int. Cl. A61B 5/16; A61B 5/00; A61B 5/0205

(52) U.S. Cl. CPC A61B 5/165; A61B 5/0002; A61B 5/021; A61B 5/681; A61B 5/7275; A61B 5/0205

(57) ABSTRACT

A monitoring system for persons at suicidal risk, the monitoring system includes a wireless network; a central monitoring station, having a computing device with a display; and a monitoring portal to receive information, the monitoring portal having an alarm to provide an alert based on the information received; a wristband having a unique identifier assigned via the central monitoring station; a control center having a power source and a transceiver to wirelessly communicate with the central monitoring station; a GPS tracking device to provide location information to the central monitoring station; a vital sensor integrally incorporated into the wristband to determine a vital sign; and a lock to secure the wristband to the person, the lock being associated with a key; the alarm function is programmed to provide the alert based on the vital sign hitting a predetermined value.

### BACKGROUND

### 1. Field of the Invention

The present invention relates generally to patient care systems for persons at risk of suicide, and more specifically, to a monitoring system for tracking locations and vitals associated with persons at risk for the prevention of suicide.

### 2. Description of Related Art

Suicide is a devastating problem associated with many government and private agency facilities, such as youth detention centers. These facilities conventionally provide housing and monitoring of person, however, the monitoring is via medical checkups, physically checking in on the persons, and visibly monitoring the persons.

One of the problems commonly associated with these types of centers is the long periods of time when the individuals are not monitored. For example, the individuals are conventionally left alone at night, providing them with ample time to cause harm to themselves.

Accordingly, it is desirable to create a system for convenient monitoring from a central location of a plurality of persons. It should be appreciated that the system of the present invention provides a means to monitor a plurality of persons from a central location and is not limited strictly to government or private agency facilities.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1 is a simplified schematic of a monitoring system in accordance with a preferred embodiment of the present application;

FIG. 2 is a simplified example of a monitoring portal of FIG. 1;

FIG. 3 is a simplified side view of a wristband of FIG. 1;

FIG. 4 is a simplified side view of an alternative embodiment of a wristband of FIG. 1;

FIG. 5 is a simplified schematic the features of the wristbands of FIG. 1; and

FIG. 6 is a flowchart of the method of use of the system of FIG. 1.

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of use in accordance with the present application overcomes one or more of the above-discussed problems commonly associated with conventional monitoring systems. Specifically, the present invention provides a means to monitor a location and vital signs of a plurality of persons from a central location, thereby allowing for a fast reaction in the event that a person's vital signs indicate dangerous conditions. These and other unique features of the system and method of use are discussed below and illustrated in the accompanying drawings.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

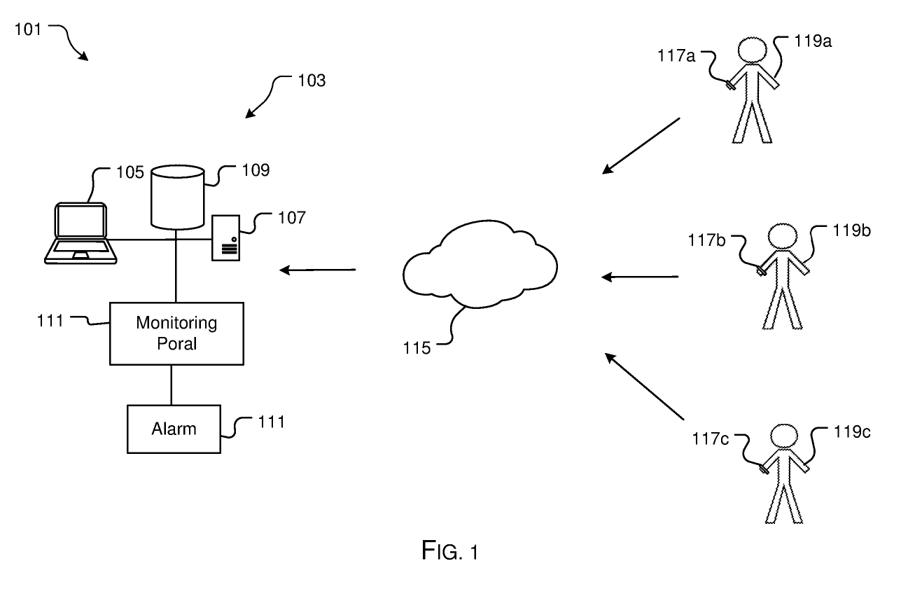

Referring now to the drawings wherein like reference characters identify corresponding or similar elements throughout the several views, FIG. 1 depicts a simplified schematic of a monitoring system 101 in accordance with a preferred embodiment of the present application. It will be appreciated that system 101 overcomes one or more of the above-listed problems commonly associated with conventional monitoring systems.

In the contemplated embodiment, system 101 includes a central monitoring station 103 having one or more of a computing device 105, a server 107, and a database 109, wherein monitoring personnel can access information through a portal 111. It should be appreciated that station 103 can be included in a single computing device accessed by one person, or alternatively, can be multiple computing devices and located within predetermined areas, such as a designated room, wherein a plurality of personnel have access to the monitoring portal. In the preferred embodiment, portal 111 is configured to provide an alarm 113 based on information receive through a network 115 from one or more wristbands 117a-c. It should be appreciated that the one or more wristbands 117a-c are assigned unique identifiers to coordinate to persons 119a-c, thereby ensuring that station 103 can distinguish information received by each wristband.

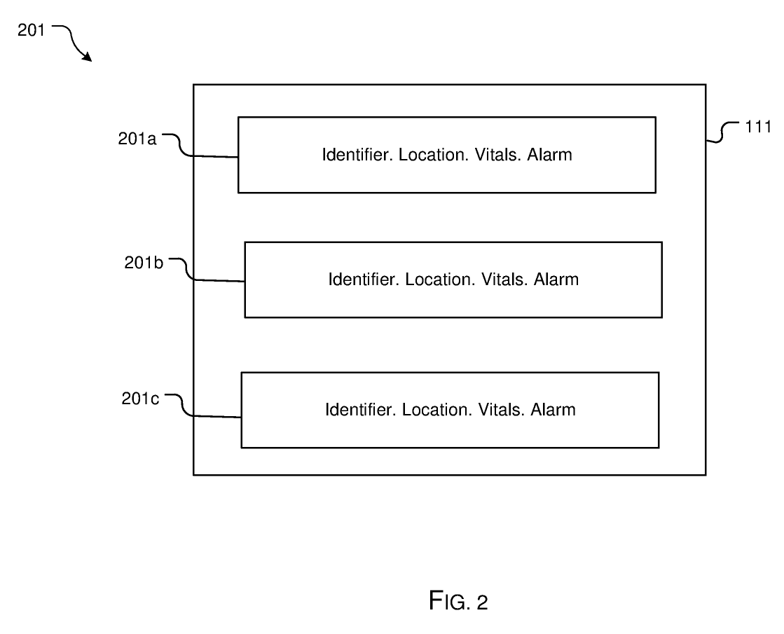

In FIG. 2, a simplified schematic 201 demonstrates features associated with portal 111. Portal 111 provides series of information 201a-c relating to persons being monitored. Further, each series 201a-c can be coordinated with an alarm, thereby providing an alert upon determination that a person's vital signs are showing distress. It should be appreciated that the alert can be visual and/or audio.

It should further be understood that the initiating of the alarm can be pre-programmed to activate upon a person's pulse dropping or rising to a predetermined value, thereby indicating stress or possible physical harm. In addition, the alarm can be programmed to activate upon a person's blood pressure rising or dropping, or their oxygen levels rising or dropping to pre-established values.

It should be appreciated that one of the unique features believed characteristic of the present application is the use of a plurality of wristbands configured to lock to a plurality of persons at risk of suicide, wherein vitals and location can be tracked via a central monitoring station.

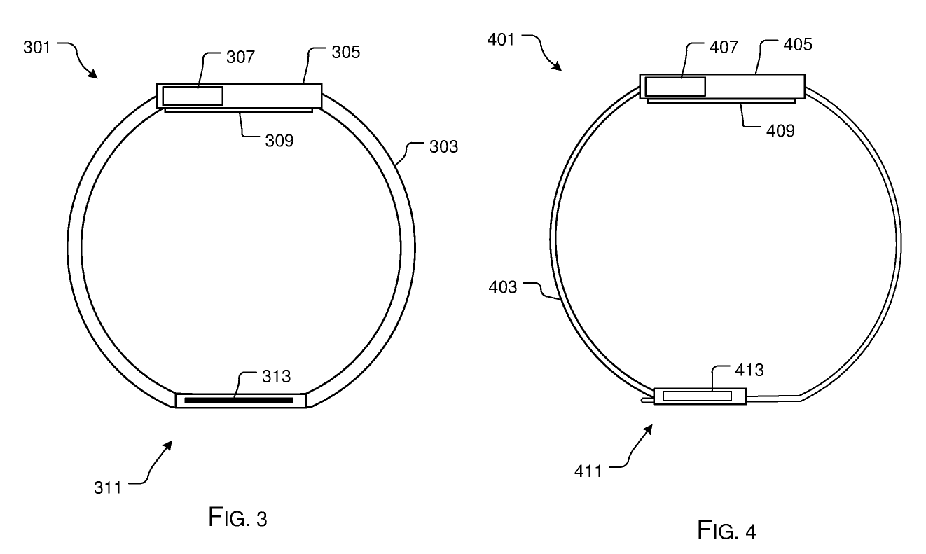

In FIG. 3, a side view of one embodiment of a wristband 301 is shown, wherein a band 303 is configured to secure to a person's wrist and is composed of a tough, durable material, such as a cut-resistance nylon, or other material having wiring or other stainless steel components, making the band difficult to tear, break, or cut. Wristband 301 further includes a housing 305 having the necessary components to communicate with central monitoring station 103 via a control center 307. Housing 305 further includes one or more vital sensors 309, such as a heart rate monitor, a blood pressure monitor, and/or a blood oxygen monitor. A lock 311 is further used to lock the wristband to the wearer, wherein the lock is only removeable by a special key associated with the central monitoring station, thereby ensuring that the wearer cannot remove. In this embodiment, a chip 313 is incorporated into the lock 311, wherein securing of the lock together activates the chip to prevent unlocking. The chip can be magnetic, thereby being removable by a magnetic key, or alternatively, the chip can be digital, wherein a command sent from the central monitoring station (or other electronic device) can be used to remove the wristband.

In FIG. 4, an alternative embodiment of a wristband 401 is shown, having a tear/cut resistant band 403 and a housing 405 with a control center 407, and a vital sensor 409. In this embodiment, a cinching lock 411 is provided wherein a physical code 413 can be implemented to release the lock 411. It should be appreciated that any locking device known in the art could be used to achieve the same features discussed herein.

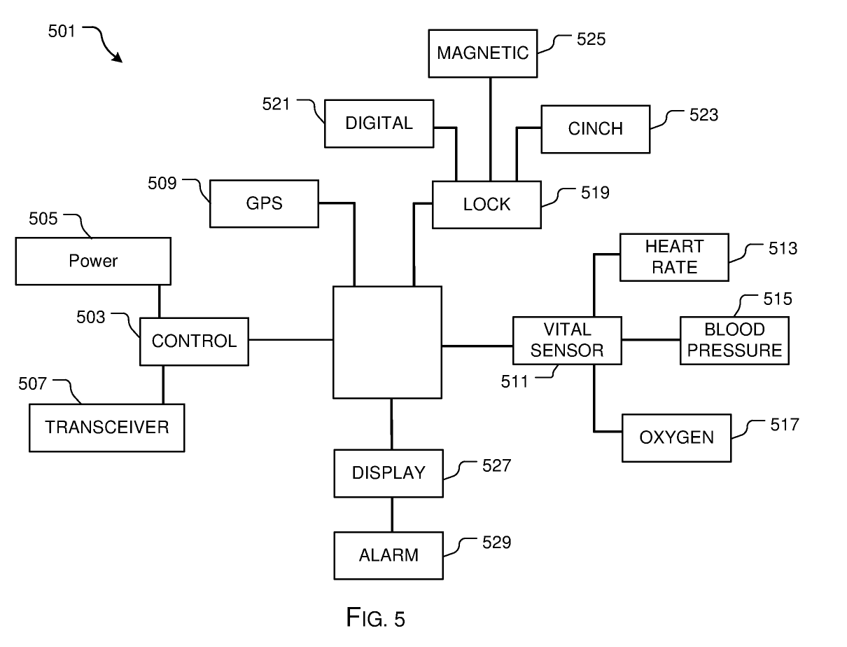

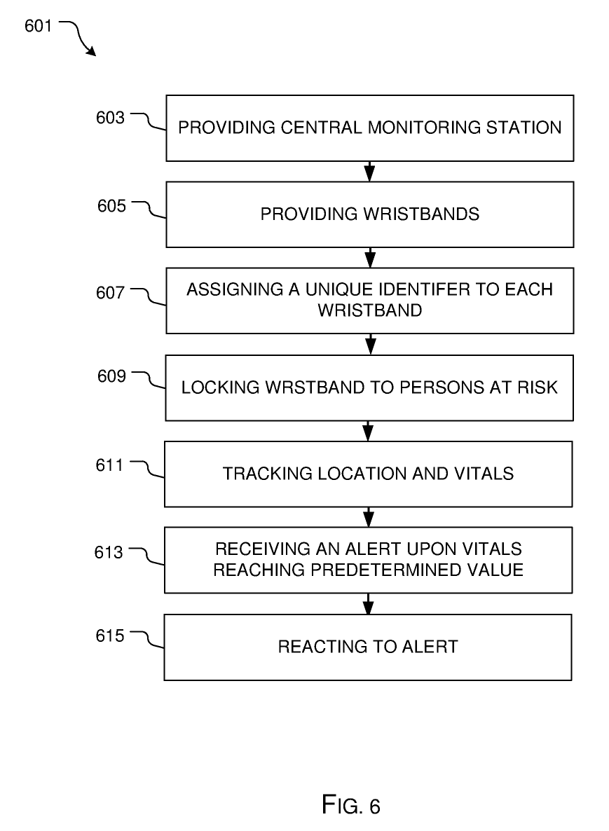

In FIG. 5, a simplified schematic 501 shows the features contemplated to be included in the plurality of wristbands included herein. Each wristband includes a control center 503 having a power source 505 and a transceiver 507, thereby being configured to communicate with the central monitoring station and provide data thereto. Each wristband further includes a GPS unit 509 configured to provide location information to the monitoring station and one or more vital sensors 511 to provide health related information. It should be appreciated that any vital sensor could be used, however, those preferred are heart rate monitors 513, blood pressure monitors 515, and blood oxygen level sensors 517. Lastly, each wristband includes a lock 519 as previously discussed herein, such as a digital lock 521, a cinching lock 523, or a magnetic lock 525. It must be understood that the locks contemplated for use require a key, whether the key be physical or a digital code, to unlock, the key being available only to designated personnel.

In some embodiments, each wristband further includes a display 527 such as a screen associated with control center 503 to provide the wearer with information such as a time, their vital signs, and any other information. Further, an alarm 529 can be provided directly with the wristband to provide an alert to the wearer upon their vitals reaching a pre-determined value.

In FIG. 6, a flowchart 601 depicts a method of use of system 101. During use, the central monitoring station is established, as shown with box 603. Each wristband is assigned a unique identifier to coordinate to a wearer, as shown with boxes 605, 607. Each wristband is locked onto the associated wearer, thereby providing a means to track their vitals and location via the central monitoring station, as shown with boxes 609, 611. If a wearer's vitals drop or rise to a pre-determined value, an alarm is activated, thereby alerting the monitoring station of danger and possible suicidal actions, as shown with box 613. The personnel can then react to the alert to check on the wearer of the wristband, as shown with box 615.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed is:

1. A monitoring system for persons at suicidal risk, the monitoring system comprising:
a wireless network;
a central monitoring station, having:
a computing device with a display; and
a monitoring portal configured to receive information regarding the persons at suicidal risk, the monitoring portal having an alarm function to provide an alert based on the information received;
a wristband having:
a unique identifier assigned via the central monitoring station, the unique identifier relating to a person assigned to the wristband;
a control center having a power source and a transceiver configured to wirelessly communicate with the central monitoring station;
a GPS tracking device configured to provide location information to the central monitoring station;
a vital sensor integrally incorporated into the wristband and configured to determine a vital sign of the person to be transmitted to the central monitoring station; and
a lock to secure the wristband to the person, the lock being associated with a key unavailable to the person;
wherein the alarm function is programmed to provide the alert based on the vital sign hitting a predetermined value.

2. The system of claim 1, wherein the vital sensor is a heart rate monitor.

3. The system of claim 1, wherein the vital sensor is a blood pressure monitor.

4. The system of claim 1, wherein the vital sensor is an oxygen level sensor.

5. The system of claim 1, wherein the lock is a cinching style lock and the key is a code known to the central monitoring station.

6. The system of claim 1, wherein the lock is a digital lock and the key is a digital code known by the central monitoring station.

7. The system of claim 1, wherein the lock is a magnetic lock and the key is a magnetic device configured to unlock the magnetic lock and located at the central monitoring station.

8. A method of monitoring a plurality of suicidal risk persons, the method comprising:
providing a central monitoring station having a computing device configured to receive data relating to the plurality of suicidal risk persons;
providing a wristband configured to wirelessly communicate with the central monitoring station, the wristband having a vital sensor configured to send vital information to the central monitoring station;
locking the wristband to a person, wherein only personnel associated with the central monitoring station have the capability of unlocking the wristband;
tracking the person via a GPS unit of the wristband;
monitoring vitals of the person via the central monitoring station; and
providing an alert upon vital signs reaching a predetermined value, the alert being presented at the central monitoring station.

9. The method of claim 8, wherein the vital sensor is a heart rate monitor.

10. The method of claim 8, wherein the vital sensor is a blood pressure monitor.

11. The system of claim 8, wherein the vital sensor is an oxygen level sensor.
